(19) United States
(12) Patent Application Publication
(10) Pub. No.: US 2011/0168180 A1
(43) Pub. Date: Jul. 14, 2011

(54) ADJUSTABLE VALVE

(75) Inventor: Gerardus Wilhelmus Lugtigheid, Spijkenisse (NL)

(73) Assignee: Emergency Pulmonary Care B.V., Voorschoten (NL)

(21) Appl. No.: 12/675,085

(22) PCT Filed: Aug. 25, 2008
(86) PCT No.: PCT/NL08/50568

§ 371 Date: Apr. 16, 2010

(30) Foreign Application Priority Data
Aug. 24, 2007 (NL) 1034284

(51) Int. Cl. A61M 16/20

(52) U.S. Cl. 128/205.14; 128/205.24

(57) ABSTRACT

Adjustable valve, comprising a valve housing and a spindle, provided with a rotary knob, which via a threaded connection is rotatably received in the valve housing, furthermore comprising a valve biased against a valve seat of the valve housing by means of a helical spring, the helical spring being coupled to an axle of the knob, so that by rotation of the knob the spring length can be set to control the closing force on the valve. The threaded connection is provided with varying pitch. The helical spring can have a non-linear spring characteristic, while the variation in the pitch of the screw thread is tuned to the spring characteristic, such that the closing force varies linearly with the rotation of the rotary knob.

The invention relates to an adjustable valve according to the introductory portion of claim 1.

The invention also relates to a valve apparatus for controlling gas pressure.

The invention further relates to a method of controlling gas pressure in a valve apparatus.

Such a valve can be used for controlling a pressure, in particular the pressure with which gas is administered to a person or animal with the aid of a respiration apparatus.

Such valves are known from practice. WO 01/66175 describes an adjustable apparatus for administering a gas where both the peak inspiratory pressure (PIP) and the positive end expiratory pressure (PEEP) and the plateau pressure during the respiratory pause can be set with such an adjustable valve. To that end, a rotary knob operates an axle which is provided with linear left- or right-handed screw thread, which cooperates with a corresponding screw thread in a valve housing, while between the rotary knob and the valve a helical spring is arranged, which helical spring can be tensioned and relaxed, respectively, by turning the rotary knob to the right and to the left, respectively. Such a valve is also referred to as APL valve (Airway Pressure Limitation).

The advantage of such a construction is that the desired pressure value is infinitely adjustable. The disadvantage thereof is that setting the pressure between minimum and maximum requires the rotary knob to be rotated by several turns. A further disadvantage is that the displacement of the rotary knob is not directly proportional to the variation of the spring tension. As a result, it is not possible in practice to provide a convenient scale from which it can be determined at a glance what pressure has been set. Also, this makes it difficult for the set pressure to be changed in a simple manner during administration of a gas.

In the use of such an adjustable valve, there is a dead volume in the valve housing and the outlet of the respiration apparatus, which needs to be pushed away by the patient before he can exhale. This dead volume may constitute a problem, especially for patients having a small lung volume, such as neonates, where the minimal force provided by the pressure—volume relationship is very quickly exhausted.

A drawback of the existing adjustable valves is further that leakage air may occur. As a result, for instance in the case of a PEEP valve, the residual pressure after expiration may slowly lessen. To prevent this, the valve is usually made of sticky design, but in this way an additional threshold for the patient is formed.

Another drawback in existing valve systems is that the gas flow through the valve housing may influence the closing pressure of the valve. As a result, for instance in the case of an APL valve, the opening pressure may deviate up to 20% from the value pre-set by the turning knob making the indicated scale value unreliable. Such pressure fluctuations may compromise the safety of patients.

The object of the invention is to provide an adjustable valve which, while preserving the advantages, prevents at least one of the disadvantages mentioned.

To that end, the invention provides a valve according to claim 1.

By providing a varying pitch, i.e. a non-linear displacement, the spring characteristic can be compensated via the configuration of the screw thread, so that the relation between rotation of the rotary knob and the variation of the closing force on the valve can be chosen more freely in design. The spring characteristic can relate to a single helical spring, but may also be the resultant of an assembly of helical springs, whereby a helical spring for closing force on the valve cooperates for instance with a helical spring for damping of the valve.

The helical spring can for instance have a non-linear spring characteristic. By tuning the variation in the pitch of the thread to the non-linear spring characteristic, it can be accomplished that the closing force nonetheless varies linearly with the displacement of the rotary knob. Preferably, the screw thread then has functionally less than one revolution, so that in practice with less than one turn of the rotary knob, for instance rotation through 270°, the closing pressure of the valve can be set linearly over the adjustment range.

Such an adjustable valve can be designed in a variety of ways allowing it to be fitted in a simple manner both in an existing apparatus for administering a gas and in a ventilation balloon (resuscitator). Preferably, the valve is so dimensioned that in open position of the valve the air can flow through the open valve freely and without restrictions. The valve according to the invention can be used to control both the peak inspiratory pressure (PIP) and the residual pressure after expiration (PEEP). Controlling the maximum pressure in accordance with the invention is more widely applicable than in respiration apparatus alone, and may also be used, for instance, for controlling the maximum pressure in gas pipes, tapping installations and the like.

In an advantageous embodiment, the adjustable valve is integrated with a non-return valve, a flow-through valve, a back pressure valve or combinations of these valves. The adjustable valve may further be integrated in the inlet port of a ventilation balloon (resuscitator).

Another object of the invention is to provide for a valve apparatus that has a relatively stable closing pressure over a relatively wide gas flow range.

This object and/or other objects may be achieved by a valve apparatus according to claim 16.

By providing the valve with a flap extending beyond the exterior of the valve seat, at least in a closed position, the lift of the valve can be increased, in particular at relatively low gas flow rates, resulting in a relatively stable closing pressure of the valve over a relatively large gas flow range.

Abovementioned object and/or other objects may also be achieved by a method of controlling gas pressure according to claim 29.

The invention will be further elucidated on the basis of exemplary embodiments which are represented in a drawing. In the drawing:

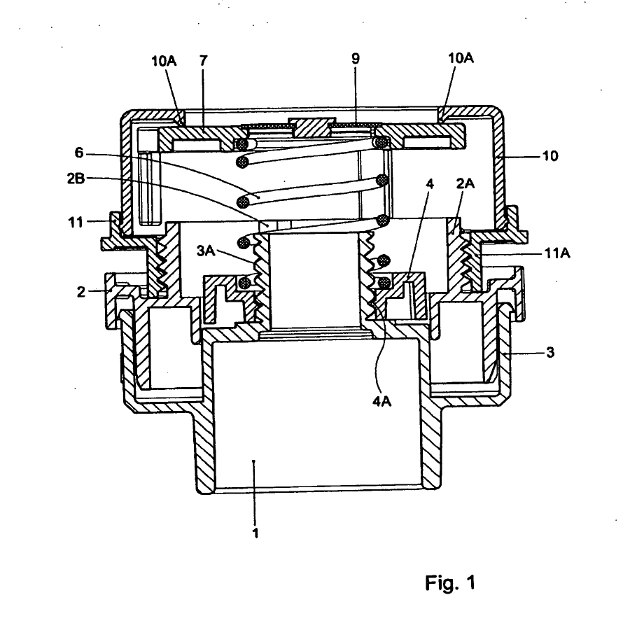

FIG. 1 shows a schematic cross section of a two-way valve with adjustable maximum pressure according to the invention.

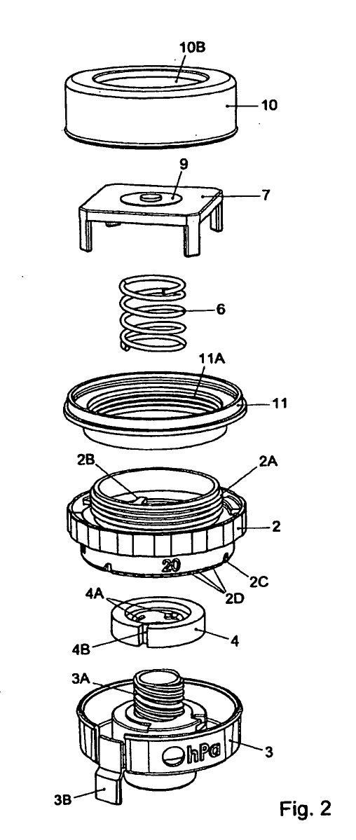

FIG. 2 shows an exploded view of the adjustable two-way valve of FIG. 1.

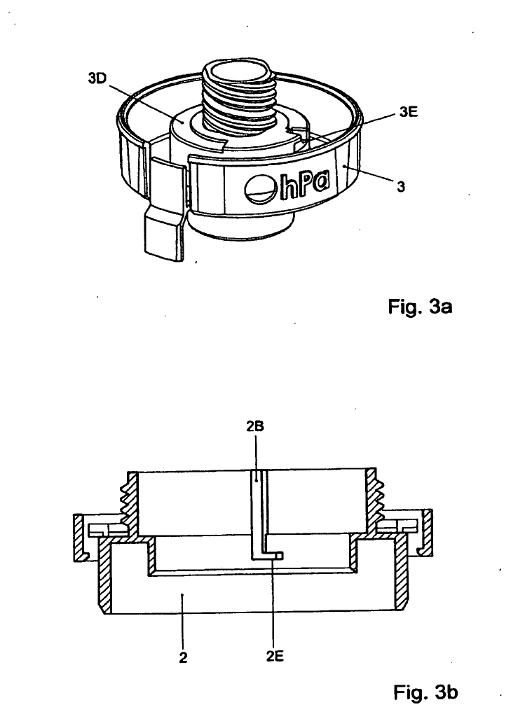

FIG. 3a shows a schematic view of a spindle with a detail of a locking mechanism of the valve.

FIG. 3b shows a schematic view of a guide with a corresponding detail of the locking mechanism of FIG. 3a.

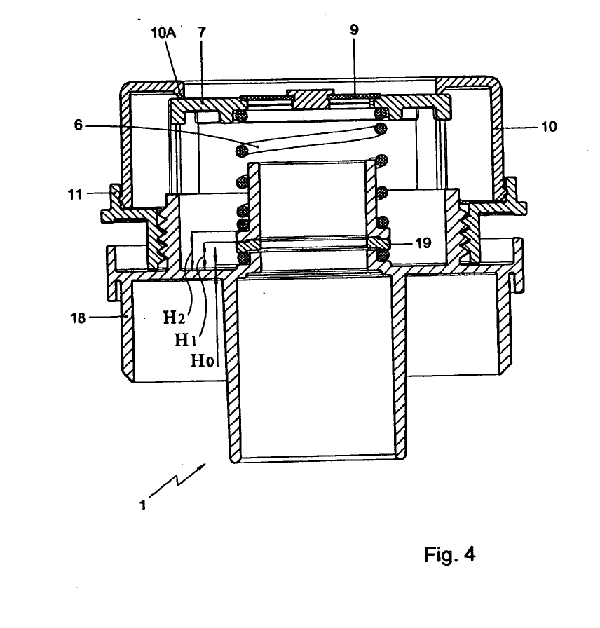

FIG. 4 shows a schematic view of a non-adjustable two-way valve.

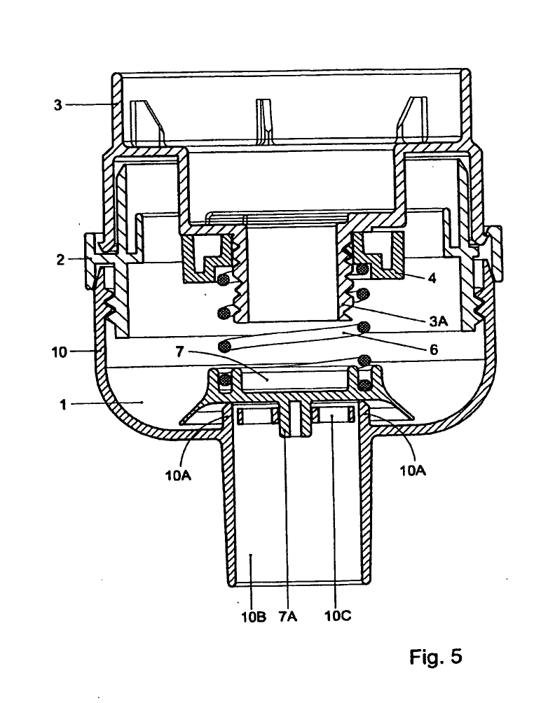

FIG. 5 shows a schematic cross section of an adjustable PEEP valve according to the invention.

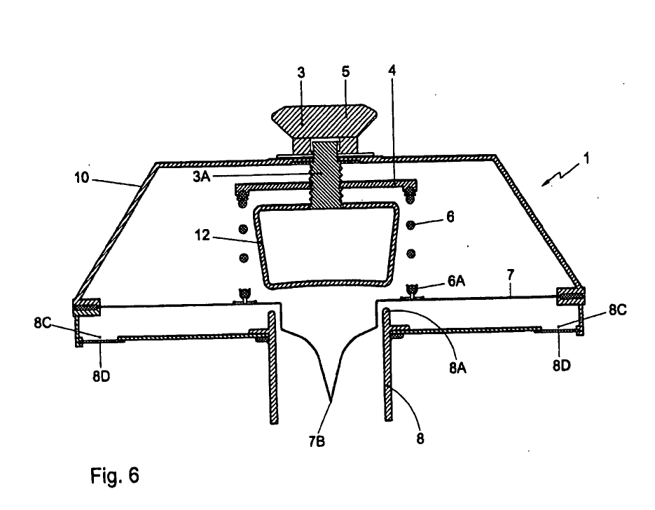

FIG. 6 shows a schematic cross section of a PEEP valve according to the invention integrated in the head of a resuscitator.

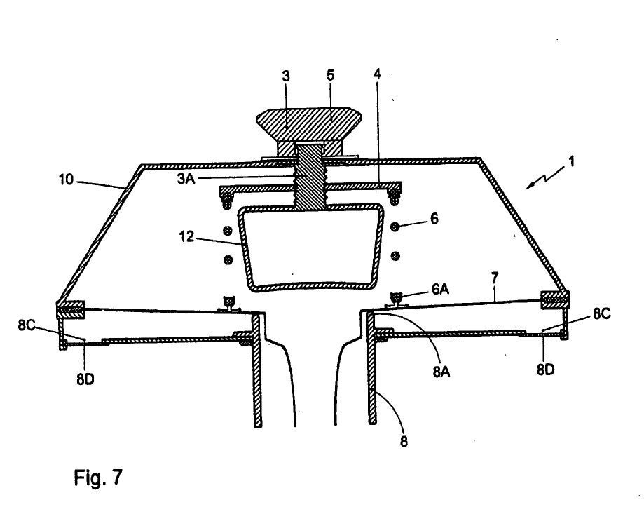

FIG. 7 shows a schematic cross section of the PEEP valve of FIG. 6 during inspiration.

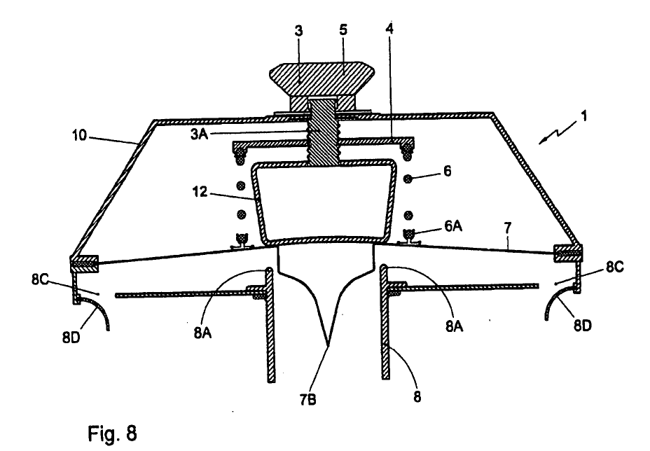

FIG. 8 shows a schematic cross section of the PEEP valve of FIG. 6 during expiration.

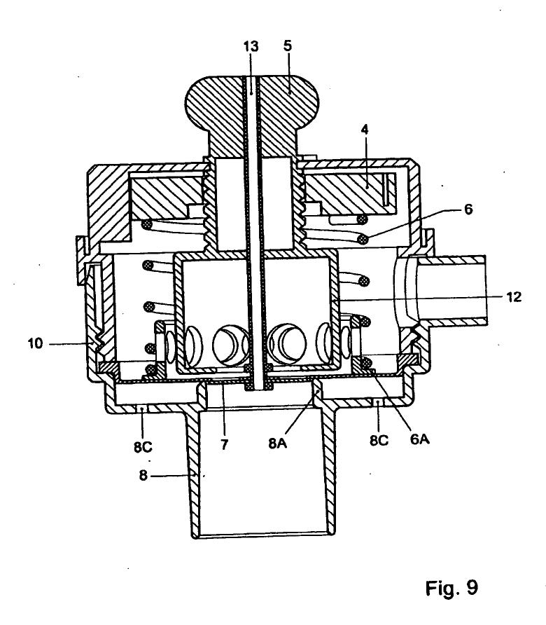

FIG. 9 shows a schematic cross section of a second embodiment of the PEEP valve.

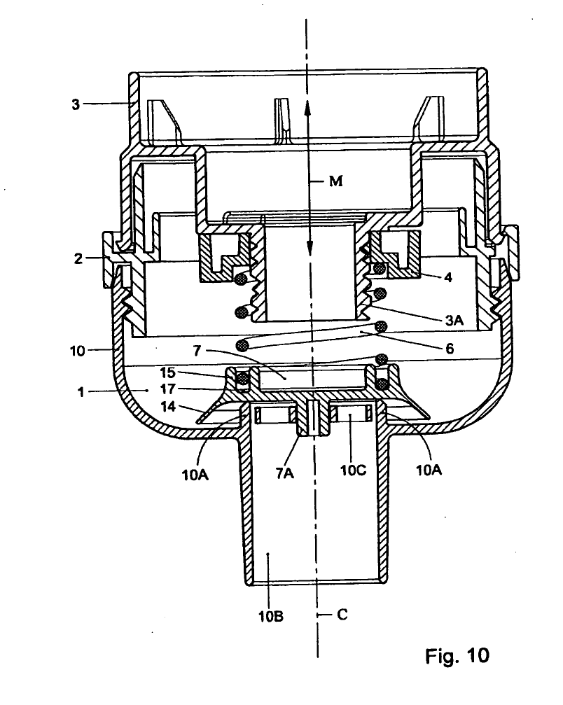

FIG. 10 shows a schematic cross section of a PEEP valve.

FIG. 11 shows a detail of the cross section of FIG. 10.

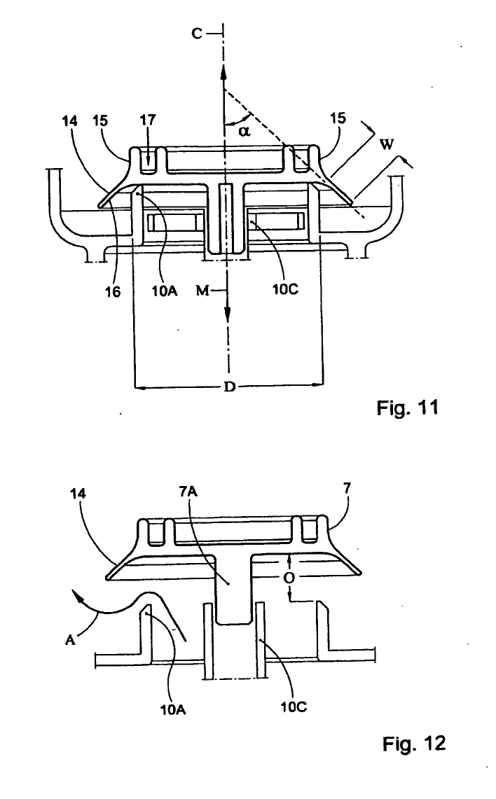

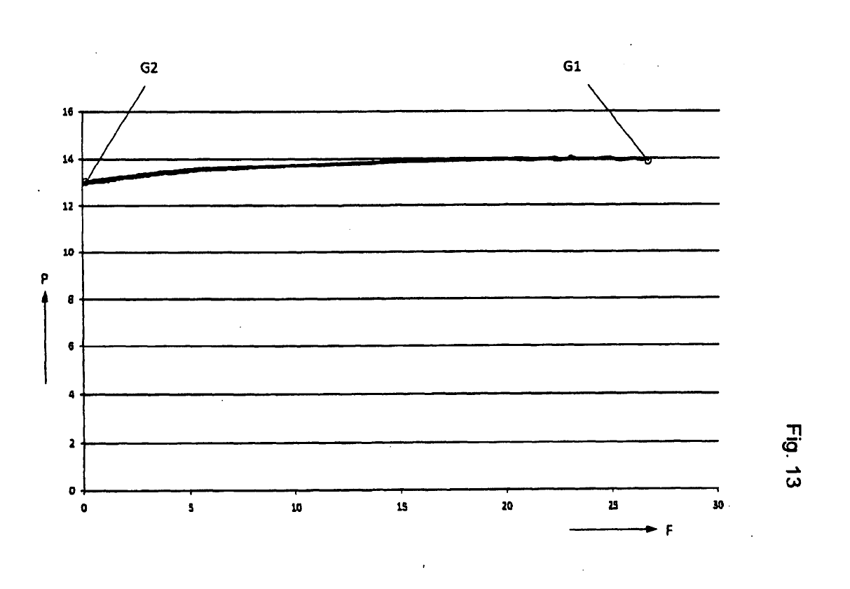

FIG. 12 shows a schematic cross section of a valve in opened condition.

FIG. 13 shows a schematic graph of the pressure on the valve plotted against the flow rate, during exhaling of a patient.

The drawings are only schematic non-limiting representations of preferred embodiments of the invention.

FIG. 1 shows an adjustable two-way valve with adjustable maximum pressure 1, comprising a valve housing 10 and a spindle 3, provided with a rotary knob 5, which, by way of a threaded connection 3A, 4A, is rotatably received in the valve housing 10. The spindle 3 is here designed with screw thread having a continuous thread 3A, and a thrust nut 4 with screw thread having an interrupted thread 4A for cooperation with the screw thread 3A.

The interrupted thread 4A here comprises a number of supports. The supports function as engagement points for cooperation with the screw thread 3A of the spindle 3. Optionally, the interrupted thread can also comprise legs or tongues. The engagement points 4A in this exemplary embodiment form guide parts to be received in grooves of the screw thread 3A cooperating therewith.

The screw thread 3A in this exemplary embodiment has a pitch varying along the thread, and has functionally less than one revolution. The screw thread 3A may be provided with multiple threads.

The spindle 3 can be rotated in a guide 2, so that the thrust nut 4 can be moved via the threaded connection comprising the mutually engaging screw thread of the spindle 3A and engagement points of the thrust nut 4A. As a result, a helical spring 6 is clamped against a valve 7, which rests on a valve seat 10A. In the valve 7, a non-return valve 9 is arranged. The helical spring 6 here has a non-linear spring characteristic and is coupled to an axle of the rotary knob 5. The axle of the rotary knob 5 here coincides with the spindle 3.

The engagement points 4A are arranged on a thrust nut 4 which is connected with the guide 2 via a linear guide 2A so as to be rectilinearly displaceable, restrained from rotation. To that end, the thrust nut 4 is provided with a guiding recess 4B which cooperates with the linear guide 2B. Via the linear guide 2B, the rotary movement of the knob 5 can be converted into a rectilinear movement for compressing, or relaxing, the helical spring 6.

The parts mentioned may for instance be manufactured from plastic material. When metal is chosen, for instance for the helical spring, for instance phosphor bronze may then be used to prevent magnetic influences. In this way, the top-piece can be used in and on the MRI.

Non-return valve 9 preferably has only its outer edge resting on valve 7 in order to prevent adhesion resulting for instance from fouling, sterilization and/or drying of moisture at the contact surface. To prevent adhesion, the valve may furthermore be designed in ceramic-coated or solid ceramic material. By setting back the boundary of the surface, the contact surface of the self-priming valve is reduced. This prevents opening forces other than reduced pressure in the balloon from playing a role.

Valve 7 is preferably designed with a relatively large diameter, so that upon opening a relatively large passage is created through which the supercompressed air can escape from the balloon with a low resistance. Moreover, the relatively large surface of the valve 7 provides for an accurate control of the inspiratory pressure that is achieved in the patient.

FIG. 2 shows an exploded view of the adjustable valve 1, visualizing the screw thread 3A of the spindle, the engagement points 4A, guiding recess 4B of the thrust nut 4 and the linear guide 2B of the guide 2.

The screw thread may be set up as a 4-fold thread with one of the threads being of wider design than the three other threads. In this way, it is possible to provide a key, so that the spindle 3 can be received in the thrust nut 4 in a single way only, for receiving the knob 5 in the valve housing 10 in a single way.

By making the four threads each of a different design, for instance with a mutually different pitch and/or a mutually different thickness, four keys can be formed. Each key then corresponds to a different scale. Each key then cooperates with a different spindle, which can be received in the thrust nut in one way only. Thus, with a single thrust nut in this example, four different spindles with corresponding scales can be fitted on a valve 1, defining four adjustable ranges each having a fixed ratio of rotary movement to rectilinear movement. The differences between the adjustable ranges are determined by the spring force of the tensioning spring and the pitch of the spindle 3. In this way, a design of a valve can be used for different applications, depending on the spindle with scale being used.

To that end, in the thrust nut 4, a number of engagement points 4A are provided, corresponding to the number of threads of the screw thread 3A and the dimensions of each of those threads. There is provided for guidance of the thrust nut 4 via a recess 4B which cooperates with a linear guide 2B arranged in the guide 2. The pitch in the threaded connection is preferably made complementary to the spring characteristic, so that a rotary clockwise displacement corresponds to a linear increase of spring force and a counterclockwise rotation corresponds to a linear decrease of the spring force. The engagement points 4A then form the positions where the thread of the thrust nut 4 is actually provided. This interrupted thread 4A provided in the thrust nut 4 prevents the non-linear screw thread 3A of the spindle 3 from jamming in it. The variation in the pitch of the threaded connection 3A, 4A is tuned to the spring characteristic, such that the closing force on the valve 7, over at least a part of the adjustment range, varies linearly with the rotation of the rotary knob 5. In an advantageous embodiment, the adjustment range of the rotary knob 5 is less than 360°, preferably circa 270°. The screw thread 3A of the spindle 3 can have less than one revolution for setting the closing pressure on the valve 7 over the whole adjustment range with complete adjustment of the rotary knob. By making the pitch over one revolution in the screw thread 3A for instance about 15 mm, the displacement of the thrust nut 4 through a rotation of the axle through 270° is approximately equal to 11.25 mm. The height of the valve housing 10 must be dimensioned to allow the thrust nut 4 to be displaced over such a distance.

The position coding realized with the key makes it possible to provide the rotary knob 5 with a scale division. On the guide 2 a scale that is directly proportional to the closing force on the valve can be provided, with a pointer on the knob 5. Also, the scale division may be provided on the spindle 3 of the knob 5, with the pointer on the guide 2. Preferably, the scale is a calibrated scale. The scale may be positioned at the top or on the side.

The threaded connection on thrust nut 4 and spindle 3 is preferably provided with left-handed screw thread, so that with a turn to the right an increasing spring load can be realized.

With a fitting guide, loss of the set pressure value through spontaneous rotation can be prevented. This is also feasible through placement of an O-ring preventing spontaneous rotation. FIG. 2 shows a practical embodiment of a protective provision against spontaneous rotation, in which the guide 2 is provided with recesses 2C between which are provided regularly spaced cams 2D. The recesses 2C and cams 2D provide a locking mechanism for locking the knob 5 at certain predefined pressure values.

The recesses 2C correspond to locked preferred settings of the valve, for instance initial position 20 hPa, intermediate positions 35 and 45 hPa, and end position 60 hPa. The distance between the recesses is divided with cams 2D into substantially equal steps of, for instance, 1 hPa. The spindle 3 is provided, on the inside thereof, with a cam (not visible in the drawing) which fits into the recess 2C, thus allowing a preferred setting of the valve to be locked. For example with the lever 3B, this lock can be removed, whereupon the spindle 3 can be rotated to a next preferred setting. The lock might also manually be removed by exerting more force as to rotate the spindle through the locked position. During rotation, the cam of the spindle 3 will come into contact with the cams 2D, so that a ratchet effect will occur which will function as an audible indicator of any desired, or spontaneous, rotation. Valve 7 can be designed as a square table cooperating with a circular valve housing 10, as e.g. shown in FIG. 2. As a result, when valve 7 is lifted from valve seat 10A, four arched slits are formed, allowing air to be released without restriction irrespective of the orientation of the adjustable valve 1.

Mounting the valve housing may be effected by means of a clamping device on the balloon. To that end, in FIG. 2 a clamping ring 11 is visible, with screw thread 11A, which can cooperate with screw thread 2A of guide 2 to clamp the outlet of the balloon between clamping ring and guide.

The adjustable valve 1 may further be provided with a locking mechanism comprising a manual override function, as shown in FIG. 3a and FIG. 3b. For example, the locking mechanism may comprise a notch 3E for cooperation with an arm 2E of the linear guide 2B. During rotation of the spindle 3, e.g. in clockwise direction, the arm 2E will move over the base 3D of the spindle 3. By pushing the spindle 3 against the spring force of the spring 6 and turning the spindle 3 in clockwise direction, the arm 2E locks into the notch 3E of the spindle. The guide 2 and the spindle 3 are then rotatably coupled, ruling out the adjustment function of the valve 1. The valve 7 is then locked in a closed position against the valve seat 10A, which may be indicated by a lock sign on the scale. The arm 2E may be disengaged from the notch 3E by twisting the spindle 3 in opposite direction, e.g. in counterclockwise direction. The locking mechanism may allow an expert user to lock the setting of the valve 7 in a closed position on valve seat 10A. When valve 7 is locked in this way, the force exerted on the balloon by e.g. the expert user may determine the inspiration pressure irrespective of the spring force of the spring 6.

The combination of thrust nut, left-handed, provided with a secure restraint from rotation, the coded multiple thread of coarse pitch providing support at several points, and the locked position of the pointer or scale on the spindle, enable a multifunctional calibrated setting which can be placed on different valve functions. Basically, this construction can be provided on any adjustable valve, such as an APL valve (Airway Pressure Limitation), or PEEP valve.

The valve 1 may also be designed as a non-adjustable valve with a fixed value. FIG. 4 shows a non-adjustable valve, comprising a valve housing and a knob, further comprising a valve biased against a valve seat of the valve housing by means of a helical spring, the helical spring via a shoulder coupled to an axle of the knob, wherein the closing force on the valve depends on the height of the shoulder. In such a non-adjustable or fixed-value valve, for instance the screw thread and thrust nut cooperating therewith may be absent. The guide 2 and the spindle 3 may then be combined to form one component 18, with the scale division omitted. On the knob 18, for instance only the pressure value is then provided. The spring 6 in the fixed-value valve rests, on a shoulder 19 of the knob 18. Depending on the height H of the shoulder 19, the spring 6 can be compressed more, or less. The height of the shoulder 19 may correspond to a particular pressure value. For example, a height H0 of the shoulder 19 may correspond with a pressure value of 20 hPa, a height H1 of the shoulder 19 may correspond with a pressure value of 35 hPa, a height H2 of the shoulder 19 may correspond with a pressure value of 45 hPa. The pressure value may be provided on the knob 18, for instance by designing the knob 18 in a particular color and/or by indicating the pressure value on the knob 18. Thus, the valve 1 has a different fixed pressure value depending on the knob 18 used. This may for instance be favorable for emergency situations when less experienced users are going to use the valve. By providing a fixed-value valve of which the pressure value depends on the height of the shoulder, a relatively simple and reliable non-adjustable valve can be provided.

FIG. 5 shows an adjustable PEEP valve 1, comprising a valve housing 10, a guide 2, a spindle 3 and a thrust nut 4. The screw thread 3A has a pitch varying along the thread, and has less than one revolution. The screw thread is here provided with multiple threads.

In the same way as explained earlier with reference to FIG. 1, the spindle 3 can be rotated in the guide 2, so that thrust nut 4 can be moved. As a result, a helical spring 6 is clamped against a valve 7, which rests on a valve seat 10A. In contrast to the non-return valve in FIG. 1, which rests on the valve seat by a smallest possible contact surface, valve 7 in this case is so designed that a relatively large contact surface is obtained with the valve seat 10A, so that the valve in closed condition seals the outflow opening 10B substantially leak-tightly, the built-up pressure thereby remaining substantially constant. What is thus achieved is that a relatively constant residual pressure (PEEP) prevails in the patient's lungs, which prevents the collapse of alveoli. Further, valve 7 is provided with a guide rod 7A which cooperates with guide 10C to prevent lateral movement of valve 7.

The parts mentioned may for instance be manufactured from plastic material. When metal is chosen, for instance for the helical spring, for instance phosphor bronze may then be used to prevent magnetic influences. In this way, the top-piece may be used in and on the MRI.

FIG. 6 shows an adjustable PEEP valve 1 at rest, which can be integrated in the head of a ventilation balloon (resuscitator) comprising a valve housing 10, a motion limiting body 12, a spindle 3 and a thrust nut 4. The screw thread 3A has a pitch varying along the thread, and has less than one functional revolution. The screw thread is here provided with multiple threads.

In the same way as explained earlier with reference to FIG. 1, the spindle 3 can be rotated, so that thrust nut 4 can be moved. As a result, a helical spring 6 by way of contact seat 6A clamps the freely movable valve 7 against a valve seat 8A, so that the channel between the patient connection 8 and the outlet opening 8C is shut off.

FIG. 7 shows that by squeezing the balloon, the pressure in the valve housing 10 rises and the beak 7B in valve 7 opens, so that air flows from the balloon to the patient. A flap valve 8D prevents reuse of exhaled air during spontaneous breathing. As soon as the balloon is no longer squeezed, the beak 7B closes and expiration can begin.

As shown in FIG. 8, during expiration, the valve 7 is clamped against motion limiting body 12 because the pressure in the lungs of the patient is higher than the force exerted on valve 7 by helical spring 6. As a result, the channel between the patient connection 8 and the outlet opening 8C and the flap valve 8D are opened and air flows out of the patient to the surroundings. As soon as the pressure in the lungs of the patient is equal to the ambient pressure, valve 7 is again clamped against valve seat 8A, and the channel between patient connection 8 and outlet opening 8C and flap valve 8D are closed. Thereupon follows a respiratory pause, after which the cycle as described above can repeat itself.

By integrating the PEEP valve 1 in the head of a ventilation balloon, the volume of the outlet channel is reduced considerably. This advantage applies especially when ventilation is done with strokes of a very small volume, as with neonates. The volume can further be reduced by providing a motion limiting body 12 which forms a closed hollow space in the valve housing 10.

The PEEP valve 1 may also be designed as a non-adjustable PEEP valve with a fixed value. To this end, in such a valve, for instance the screw thread and thrust nut cooperating therewith may be absent. The motion limiting body 12 and the spindle 3 may then be combined to form one component, with the scale division omitted. On such a combined spindle, for instance only the PEEP value is then provided. The spring in a fixed-value PEEP valve rests, for instance, on a shoulder of the spindle, or on a spring seat. Depending on the height of the shoulder, the spring can be compressed more, or less. The height of the shoulder corresponds for instance to a particular pressure value. The pressure value can be provided on the spindle, for instance by designing the spindle in a particular color and/or by indicating the PEEP value on the spindle. Thus, the PEEP valve has a different fixed PEEP value depending on the spindle used. This may for instance be favorable for emergency situations when less experienced users are going to use the PEEP valve. The invention concerning the non-adjustable valve is not limited to the exemplary embodiments of a non-adjustable two-way valve and a non-adjustable PEEP valve. Many variants are possible.

In FIG. 9 a second exemplary embodiment of an adjustable PEEP valve 1 is shown. The motion limiting body 12 in this exemplary embodiment is provided with recesses to further reduce the volume of the outlet channel.

Furthermore, in this exemplary embodiment, a measuring tube 13 is provided to measure the pressure. In this way, for instance, it may be verified whether the pressure set is actually achieved. The measuring tube 13 extends through the valve 7 which is here designed as a ring-shaped valve which is supported adjacent the middle by the measuring tube 13. The measuring tube 13 may also be used to measure the CO₂ content.

In practice, for instance, different types of valves with a different adjustment range may be provided. Thus, APL valves will for instance be adjustable between circa 20-60 hPa and 40-120 hPa, respectively. PEEP valves will for instance be adjustable between circa 0-20 hPa. Advantageously, parts of the different types of valve may then be purposely made of incompatible design to preclude improper assembly.

In FIG. 10 another embodiment of a valve apparatus for controlling gas pressure is shown, similar to the embodiment shown in FIG. 5. In particular, a part of a respiration apparatus for administering gas to a patient is shown. The valve apparatus comprises a valve housing 10 and a valve 7, which valve 7 is supported by a valve seat 10A, at least in a closed position. The valve seat 10A may have a circumferential shape. Preferably, the valve 7 is provided with a flap 14, formed by a circumferential flange at the outer edge of the valve 7. A guide element 7A, in particular a rod, is provided for guiding the valve 7 between an open and closed position, in a main direction of movement M of the valve 7. The guide element 7A may for example extend through and/or parallel to a central axis C of the valve 7. The guide element 7A may cooperate with a corresponding guide 10C that is provided in the valve housing 10. The guide element 7A and the corresponding guide 10C are arranged so that the valve 7 opens and closes along a straight direction, more particularly in said main direction of movement M. As already explained above, a spring 6, preferably a helical spring, may be provided by which the valve 7 is spring biased in the direction of the valve seat 10A. By adjusting spring pressure, for example by using the spindle 3, pressure on the valve 7 can be adjusted.

The valve 7 and the valve seat 10 A are shown in more detail in FIG. 11. As can be seen the valve 7 comprises a flap 14 that extends beyond the exterior of the valve seat 10A in particular next to the outside of an outer rim 15 of the spring seat 17, at least when the valve 7 is in a closed position. The flap 14 may be curved in a direction towards the closed position of the valve 7, so that the flap 14 extends next to the outside of the valve seat 10A. The inner surface 16 of the flap 14 may extend under an angle α of between about 30 and about 85°, particularly between about 45 and about 80°, more particularly between about 55 and about 75° and preferably about 65° with respect to a main direction of movement M between a closed and an open condition of the valve 7. Preferably, a relatively large contact surface is provided between the valve 7 and the valve seat 10A so that in a closed position a substantially gas tight closure may be obtained.

In an embodiment, the valve 7 preferably comprises a spring seat 17 for engaging the spring 6. Preferably, the spring 6 is at least slightly clamped or pressed in the spring seat 17, for example between an outer rim 15 and an inner rim of the spring seat 17.

In an embodiment the diameter D of the valve 7, excluding the flap 14, may for example be approximately between 5 and 80 millimeter, particularly between 10 and 60 millimeter, more particularly between 15 and 45 millimeter, and preferably approximately 30 millimeter. This diameter D may be approximately equal to the diameter of the outer rim 15 of the valve seat 10A, for example. The flap 14 may for example have a width W of between 1 and 20 millimeter, particularly between 1.5 and 10 millimeter, more particularly between 2 and 6 millimeter, and preferably of approximately 3 millimeter. The inner surface of flap 14 may extend from the perimeter of valve 7 in a straight line and at a pre-set preferred angle α, or may gradually curve in a downward direction until the preferred angle α is reached.

In an embodiment the inner surface of flap 14 is provided with a particular surface finish, such as a polished surface finish or a relatively flexible surface finish, such that the inner surface of flap 14 tightly closes on the valve seat 10A.

The lifting properties of the valve 7 may be proportional to the area provided by flap 14 and the gas velocity along the flap 14. In an open position of the valve 7, the gas flow along the flap 14 produces a lifting force that prevents the valve 7 to close on valve seat 10A. The gas flow is subjected to a flow resistance, which may be mainly proportional to the area of the flow opening O between the valve 7 and the valve seat 10A, and the bending A of the gas flow through the valve housing 10, as indicated in FIG. 12. The particular bending shape of the gas flow bending A may be influenced by abovementioned angle α of the flap. The valve 7 has shown to have relatively stable lifting properties, especially in case the lifting force provided by the flap 14 is well balanced by the flow resistance provided by the outflow opening O and the bending A of the gas flow. As a result, fluctuations in closing pressure may be limited.

As shown in FIG. 12, the flap 14 may allow for gas to flow along the valve 7 through the opening O. At relatively low flow rates the outflow opening O will be relatively narrow and may act as a throat or venturi, increasing the velocity along the flap 14, thereby increasing the lifting force exerted on the valve 7. As a result, the lifting force exerted on the valve 7 may become relatively independent of the fluid flow rate, preventing oscillations of the valve 7, thus providing a relatively continuous and better controllable closing pressure. The flow resistance may remain substantially the same at low as well as high flow rates, so that even at relatively low pressure values, fluctuations in pressure may be limited. Due to reduced fluctuations, also noise that may be caused by the valve 7, for example by ticking against the valve seat 10A at low flow rates, may be reduced. The angle α of the flap 14 may for example be chosen to be similar to the angles of e.g. wing flaps of airplanes and/or hang glider wings for providing a continuous stable lifting force to the flying body at a relatively low velocity, and preventing pitching of the flying body.

FIG. 13 illustrates a simplified, exemplary graph, wherein the pressure P on the valve 7 and the corresponding gas flow rate F through the valve 7 are plotted during an exhaling action of a patient. The pressure P is indicated by the vertical axis, and the flow rate F is indicated by the horizontal axis. The moment the exhaling action starts, the pressure P on the valve 7 may be zero, while the flow rate F is also zero. During the exhalation the flow rate F rapidly increases to a peak value while the pressure P builds up, for example up to starting point G1. From G1 towards the end point G2 of the exhaling action, the flow rate F may decrease towards zero, while the pressure P may remain relatively constant, which is illustrated by the relatively flat straight line between starting point G1 and the end point G2. Thus, a relatively flow independent closing pressure P of the valve 7 is obtained. Ideally, when the flow rate F comes close to zero, or is approximately zero, the valve 7 will close. Merely for purpose of illustration, the respective pressures P and flow rates F in the graph may for example correspond to approximately 14 hPa (hectoPascal) and approximately 28 L/Min (litres per minute) for the starting point G1, and approximately 13 hPa and approximately 0 L/Min for the end point G2.

The valve 7 may for example be of relatively light design. For example, the valve 7 may be made of a plastic and may be rigid and/or flexible, for example partly rigid and partly flexible. The valve 7 may for example be provided with a flexible part so that a substantially fluid tight sealing may be obtained when the valve 7 is in a closed position. The flexible part may provide for a larger contact surface between the valve 7 and the valve seat 10A. The flexible part may comprise a sealing element such as a sealing ring and may be integrally molded with the valve 7. With such a flexible part, air leakage between the valve 7 and the valve seat 10A may be prevented.

The valve 7 with the flap 14 may be suitable for any application, in particular for gas flow controlling applications. More in particular, due to its controlled pressure capabilities at relatively low flow rates, it may be applied in a respiration apparatus.

The invention is not limited to the exemplary embodiments described here. Many variations are possible within the scope of the invention as defined in the following claims.

1. An adjustable valve comprising:
a valve housing and a spindle, provided with a rotary knob, which via a threaded connection is rotatably received in the valve housing, the adjustable valve furthermore comprising a valve biased against a valve seat of the valve housing by means of a helical spring, the helical spring being coupled to an axle of the knob, so that by rotation of the knob the helical spring length can be set to control a closing force of the valve, wherein the threaded connection has a varying pitch.

2. The adjustable valve according to claim 1, wherein the helical spring has a non-linear spring characteristic.

3. The adjustable valve according to claim 2, wherein variation in the varying pitch of the threaded connection is tuned to the non-linear spring characteristic, such that the closing force on the valve, over at least a part of an adjustment range, varies linearly with rotation of the rotary knob.

4. The adjustable valve according to claim 1, wherein the threaded connection comprises a continuous thread which cooperates with an interrupted thread.

5. The adjustable valve according to claim 4, wherein the interrupted thread comprises a number of supports.

6. The adjustable valve according to claim 4, wherein the interrupted screw thread comprises guiding parts for being received in grooves of the screw thread cooperating therewith.

7. The adjustable valve according to claim 1, wherein the valve housing comprises a linear guide for converting a rotary movement of the knob into a rectilinear movement for compressing, or relaxing, the helical spring.

8. The adjustable valve according to claim 1, wherein the threaded connection comprises a key for receiving the rotary knob in the valve housing in a single manner.

9. The adjustable valve according to claim 1, wherein the rotary knob includes a scale division, a scale of which is directly proportional to the closing force on the valve.

10. The adjustable valve according to claim 1, wherein a whole adjustment range of the rotary knob is less than 360°.

11. The adjustable valve according to claim 10, wherein the threaded connection has less than one revolution for setting the closing pressure on the valve over the whole adjustment range with complete adjustment of the rotary knob.

12. The adjustable valve according to claim 1, wherein at least a part of multiple parts of the threaded connection is provided with multiple threads.

13. The adjustable valve according to claim 1, wherein a screw thread in the threaded connection is designed as a left-handed screw thread.

14. The adjustable valve according to claim 1, wherein the valve is integrated in a head of a ventilation balloon.

15. The adjustable valve according to claim 14, wherein a motion limiting body is provided in the valve housing.

16. A valve apparatus for controlling gas pressure, the valve apparatus comprising:
a valve housing, provided with a valve seat; and
a valve, wherein the valve comprises a flap that in a closed position extends beyond an exterior of the valve seat.

17. The valve apparatus according to claims 16, wherein in a closed position the flap extends next to the exterior of the valve seat.

18. The valve apparatus according to claim 16, wherein the flap is formed by a circumferential ridge-shaped body extending from a perimeter of the valve.

19. The valve apparatus according any of claim 16, wherein an inner surface of the flap extends at an angle of between 30 and 85° with respect to a main direction of movement between a closed and an open condition of the valve.

20. The valve apparatus according to claim 16, wherein a spring is provided by which the valve is biased in a direction of the valve seat.

21. The valve apparatus according to claim 20, wherein the closing pressure is adjustable by adjusting a force of the spring.

22. The valve apparatus according to claim 16, wherein the valve comprises a seat for engaging a helical spring.

23. The valve apparatus according to claim 22, wherein the helical spring is clamped in the valve.

24. The valve apparatus according to claim 16, wherein the valve comprises a guide element for guiding the valve in a substantially straight direction of movement.

25. The valve apparatus according to claim 24, wherein the guide element comprises a rod approximately in and/or through a middle of the valve.

26. The valve apparatus according to claim 16, wherein a relatively large contact surface is provided between the valve and the valve seat so that in a closed position a substantially gas tight closure is obtained.

27. The valve apparatus according to claim 16, wherein the valve comprises a flexible element arranged to contact the valve seat in a closed position.

28. The valve apparatus according to claim 16, wherein the valve apparatus is a respiration apparatus for administering gas to a patient.

29. The valve apparatus according to claim 16, wherein the valve apparatus is integrated in a head of a ventilation balloon.

30. The valve apparatus according to claim 29, wherein in the valve housing a motion limiting body is provided.

31. (canceled)

32. A method of controlling gas pressure in a valve apparatus, wherein gas flows between a valve and a valve seat, along a flap of the valve that extends beyond the exterior of the valve seat.
